(19) United States
(12) Patent Application Publication
(10) Pub. No.: US 2008/0132529 A1
(43) Pub. Date: Jun. 5, 2008

(54) Method of improving bioavailability for non-sedating barbiturates

(76) Inventors: Avraham Yacobi, Englewood, NJ (US); Daniel A. Moros, Larchmont, NY (US); Derek A. Ganes, Toronto (CA)

Correspondence Address: TARO PHARMACEUTICALS U.S.A., INC.;C/O VENABLE LLP, P.O. BOX 34385, WASHINGTON, DC, 20043-9998, US

(21) Appl. No.: 11/984,226

(22) Filed: Nov. 14, 2007

Related U.S. Application Data
(60) Provisional application No. 60/858,701, filed on Nov. 14, 2006.

(51) Int. Cl. A61K 31/515

(52) U.S. Cl. 514/270

(57) ABSTRACT

Administration of 1,3-dimethoxymethyl-5,5-diphenylbarbituric acid and its related compounds with food increases the bioavailability of these compounds. The ratios (fasting/fed) for geometric mean AUC₀₋ₜ were 35.6% (DMMDPB), 36.6% (MMMDPB) and 65.3% (DPB) and the geometric mean Cₘₐₓ were 14.6% (DMMDPB), 31.9% (MMMDPB) and 62.9% (DPB). The cyclic ureides may be administered to a subject before or after ingestion of food within a defined time period.

This application claims the benefit of provisional Application No. 60/858,701, filed Nov. 14, 2006.

### FIELD OF THE INVENTION

The invention relates to methods for increasing the bioavailability of a group of cyclic ureides, such as, 1,3-dimethoxymethyl-5,5-diphenylbarbituric acid as well as its derivatives.

### BACKGROUND OF THE INVENTION

1,3-dimethoxymethyl-5,5-diphenylbarbituric acid is a cyclic ureide. It is a barbiturate which is a class of drugs that has been in wide clinical use for over one hundred years. Other members of this class have been used as sedative-hypnotics, anesthetic agents and antiepileptic agents, but in all cases sedation accompanies the pharmacological activity. (Goodman & Gilman's, The Pharmacological Basis of Therapeutics, 9^{th} Edition, pp. 373-380 (1996)). In contrast, 1,3-dimethoxymethyl-5,5-diphenylbarbituric acid and its metabolites retain useful pharmacological activities at dosages that are not accompanied by sedation. Commonly owned U.S. Pat. No. 4,628,056. In animal studies, 1,3-dimethoxymethyl-5,5-diphenylbarbituric acid and its principal active metabolite, 5,5-diphenylbarbituric acid (DPB) were found to exert anticonvulsant properties against both electroshock and chemoconvulsive seizures at doses which exhibited no neurological impairment. Commonly owned U.S. Pat. No. 6,093,820 to Gutman et al., reissued as RE 38934 on Jan. 10, 2006 discloses that another metabolite, N-methoxymethyl ethosuximide, N-methoxymethyl glutethimide, and N-methoxymethyl-5,5-diphenylbarbituric acid which are also useful in treating convulsions, seizures, muscle stiffness, or anxiety.

Pharmacokinetic studies have not previously been conducted to evaluate the effect of food on the pharmacokinetics of 1,3-dimethoxymethyl-5,5-diphenylbarbituric acid. The hydrophobicity of the 1,3-dimethoxymethyl-5,5-diphenylbarbituric acid molecule and the dosage amount required for a therapeutic effect both point to the fact that absorption from the gut is limited when the drug is administered orally. Studies with other barbiturate such as amobarbital and phenobarbital have indicated that administration with food significantly decreased the serum and brain levels of these barbiturates (Kojima, Chem. Pharm. Bull. 21(11):2432-2437 (1973); Kojima et al., J. Pharm Sci. 60:1639-1641 (1971); see also, Goodman & Gilman's, The Pharmacological Basis of Therapeutics, 9^{th} Edition, pp. 377 (1996)).

Because of the potential broad therapeutic use for the cyclic ureides of this invention, it is important to establish means for increasing the oral bioavailability of the drug substance. Specifically, there is a need to increase both the speed of onset and the extent of therapeutic effect of 1,3-dimethoxymethyl-5,5-diphenylbarbituric acid and its related compounds.

### SUMMARY OF THE INVENTION

The present invention provides a method of improving bioavailability of a pharmaceutical composition comprising administering a therapeutically effective amount of

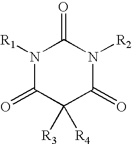

at least one compound having the following formula:
to a mammal, wherein R₃ and R⁴ are each independently selected from the group consisting of lower alkyl, phenyl and lower alkyl substituted phenyl, and R₁ and R₂ are each independently either a hydrogen atom or a radical of the formula

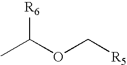

wherein R₅ and R₆ are each independently selected from the group consisting of H, lower alkyl, phenyl and lower alkyl substituted phenyl, its pharmaceutically acceptable salts and prodrugs thereof and at least one pharmaceutically acceptable excipient.

In another exemplary embodiment, the present invention provides a method of improving bioavailability of a pharmaceutical composition comprising administering a therapeutically effective amount of at least one compound having the following formula to a mammal:

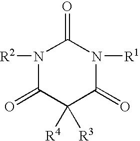

wherein R^{1 and R}² may be the same or different and are independently

lower alkyl, substituted by lower cycloalkyl, acyl, acyloxy, aryl, aryloxy, thioalkyl or thioaryl, amino, alkylamino, dialkylamino, or one or more halogen atoms;

phenyl;

C(O)XR⁶, wherein X is S or O and R⁶ is lower alkyl or aryl;

CXR⁷, wherein X is as defined above and R⁷ is hydrogen, lower alkyl or aryl; and

CH(XR⁸)₂, wherein X is as defined above and R⁸ is a lower alkyl group; and wherein

R³ and R⁴ may be the same or different and are independently hydrogen;

aryl optionally containing one or more heteroatoms selected from the group consisting of N, S, and O;

lower acyloxy;

phenyl substituted with lower acyl group or derivative thereof or acetamide; benzyl; benzyl substituted on the ring by one or more halogens, lower alkyl groups or both; cycloalkyl, which optionally contains one or more heteroatoms selected from the group consisting of N, O, and S;

lower alkyl; or lower alkyl substituted with an aromatic moiety;

provided that at least one of R³ and R⁴ is an aromatic ring or an aromatic ring containing moiety,

and salts thereof,

with the proviso that:

when one of R³ and R⁴ is benzyl, the other of R³ and R⁴ is not ethyl, the compound is other than

a) 1-methyl-5-(1-phenylethyl)-5-propionyloxy-barbituric acid,

b) 1,3-diphenyl-5,5-(dibenzyl) barbituric acid,

c) 1,3,5-triphenyl barbituric acid, and

d) 5-benzyl-1,3-dimethyl barbituric acid

In another exemplary embodiment, the present invention provides a method of improving bioavailability of a pharmaceutical composition comprising administering a therapeutically effective amount of sodium 5,5-diphenyl barbiturate or a derivative thereof and at least one pharmaceutically acceptable excipient.

The food is ingested from about 2 hours prior to administration of the pharmaceutical composition to about 2 hours after administration of the pharmaceutical composition. In one embodiment, the food may be ingested from about 2 hours to about 5 minutes before administration of the pharmaceutical composition. In another embodiment, the food may be ingested from about 30 minutes to about 5 minutes before administration of the pharmaceutical composition.

Alternatively, the food may be ingested from about 5 minutes to about 2 hours after administration of the pharmaceutical composition or from about 5 minutes to about 30 minutes after administration of the pharmaceutical composition.

After administration of the pharmaceutical composition with food, the fasting/fed ratios for the geometric mean AUC₀₋ₜ d of the cyclic ureides may be as follows: (i) DMMDPB at least about 30%; (ii) MMMDPB at least about 30%; and (iii) DPB at least about 60%. As used herein, administration of the pharmaceutical composition with food refers to the fact that the food may be ingested either before or after administration of the pharmaceutical composition. In another embodiment, the fasting/fed ratios for the geometric mean AUC₀₋ₜ d for DMMDPB is at least about 35.0%, MMMDPB is at least about 36% and DPB is at least about 65%. In yet a third embodiment, the geometric mean Cₘₐₓ for fasting/fed is at least about 14.0% for DMMDPB, at least about 31.0% for MMMDPB and at least about 62.0% for DPB.

The invention also encompasses an article of manufacture which comprises a container containing a pharmaceutical composition of a therapeutically effective amount of at least one compound according to the following formula:

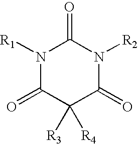

wherein R₃ and R⁴ are each independently selected from the group consisting of lower alkyl, phenyl and lower alkyl substituted phenyl, and R₁ and R₂ are each independently either a hydrogen atom or a radical of the formula

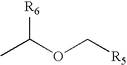

wherein R₅ and R⁶ are each independently selected from the group consisting of H, lower alkyl, phenyl and lower alkyl substituted phenyl, its pharmaceutically acceptable salts and prodrugs thereof and at least one pharmaceutically acceptable excipient, wherein the container is associated with printed labeling advising that taking the pharmaceutical composition with food increases the bioavailability to a patient receiving the composition by oral administration.

### BRIEF DESCRIPTION OF THE DRAWINGS

FIG. 1A shows the mean plasma concentration of DMMDPB (T2000), MMMDPB and DPB after single 400 mg oral doses of T2000 in fasting, healthy patients (n=23).

FIG. 1B shows the mean plasma concentration of DMMDPB (T2000), MMMDPB and DPB after single 400 mg oral doses of T2000 in fed, healthy patients (n=23).

### DETAILED DESCRIPTION OF THE INVENTION

“DMMDPB” is 1,3-dimethoxymethyl-5,5-diphenyl barbituric acid (DMMDPB may also be referred to as “T2000”); “MMMDPB” is monomethoxymethyl-5,5-diphenyl barbituric acid; “DPB” is 5,5-diphenyl barbituric acid.

For the purposes of the present invention “bioavailability” of a drug is defined as both the relative amount of drug from an administered dosage form which enters the systemic circulation and the rate at which the drug appears in the blood stream. Bioavailability is largely reflected by AUC, which is governed by at least 3 factors: (i) absorption which controls bioavailability, followed by (ii) its tissue re-distribution and (iii) elimination (metabolic degradation plus renal and other mechanisms).

“AUC” refers to the mean area under the plasma concentration-time curve; “AUC₀₋ₜ” refers to area under the concentration-time curve from time zero to the time of the last sample collection; “AUC₀₋₂₄” refers to area under the concentration-time curve from time zero to 24 hours; “AUC₀₋₄₈” refers to area under the concentration-time curve from time zero to 48 hours; “Cₘₐₓ” refers to maximum observed plasma concentration; “Tₘₐₓ” (or “tₘₐₓ”) refers to the time to achieve the Cₘₐₓ; “t_{1/2}” refers to the apparent half-life and is calculated as (ln 2/Kₑₗ), where Kₑₗ refers to the apparent first-order elimination rate constant “absolute bioavailability” is the extent or fraction of drug absorbed upon extravascular administration in comparison to the dose size administered.

“Absolute bioavailability” is estimated by taking into consideration tissue re-distribution and biotransformation (i.e., elimination) which can be estimated in turn via intravenous administration of the drug. “Improved bioavailability” refers to a higher, observable AUC, i.e., when DMMDPB is administered with food. Unless otherwise indicated, “mean plasma concentration” and “plasma concentration” are used herein interchangeably; “HPLC” refers to high performance liquid chromatography; “pharmaceutically acceptable” refers to physiologically tolerable materials, which do not typically produce an allergic or other untoward reaction, such as gastric upset, dizziness and the like, when administered to a mammal; “mammal” refers to a class of higher vertebrates comprising man and all other animals that nourish their young with milk secreted by mammary glands and have the skin usually more or less covered with hair; and “treating” is intended to encompass relieving, alleviating or eliminating at least one symptom of a disease(s) in a mammal.

Cyclic ureides used in the present invention having the following general formula:

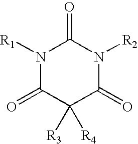

wherein R₃ and R⁴ are each independently selected from the group consisting of lower alkyl, phenyl and lower alkyl substituted phenyl, and R₁ and R₂ are each independently either a hydrogen atom or a radical of the formula

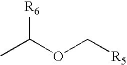

wherein R₅ and R⁶ are each independently selected from the group consisting of H, lower alkyl, phenyl and lower alkyl substituted phenyl, its pharmaceutically acceptable salts, metabolites, and prodrugs (commonly owned U.S. patent application Ser. No. 10/735,514, now issued as U.S. Pat. No. 7,166,610 on Jan. 23, 2007). Suitable prodrugs of MMMDPB include, but are not limited to, mono- and di-phosphate and mono and di-phosphonooxyalkyl derivatives of MMMDPB. Preferred prodrugs are the mono- and di-phosphonooxymethyl derivatives. One preferred type of cyclic ureides useful in the methods and compositions of the present invention are barbituric acid derivatives disubstituted at the 5-position. Another preferred embodiment of the present invention uses 5,5-diphenyl barbituric acid. Specific preferred compounds useful in the treatment method of the present invention include N,N-dimethoxymethyl diphenyl barbituric acid (DMMDPB), monomethoxymethyl diphenyl barbituric acid (MMMDPB) and diphenyl barbituric acid (DPB) as well as pharmaceutically acceptable salts and prodrugs thereof.

Cyclic ureides useful in the treatment methods of the present invention can be made by any known synthetic technique. By way of illustration, U.S. Pat. No. 4,628,056, U.S. Reissue Pat. No. RE 38934, and U.S. Pat. No. 7,166,610, herein incorporated in their entirety by reference, disclose examples of such methods for the preparation of compounds used in the present invention.

The cyclic ureides of the present invention may be formulated into pharmaceutical compositions or formulations that additionally and optionally comprise any suitable adjuvants, excipients, additives, carriers, solvents, additional therapeutic agents (e.g., for conjoint use as a combination treatment, including for example one or more additional agents), bioavailability enhancers, side-effect suppressing constituents, or other ingredients that do not adversely affect the efficacy of the pharmaceutical composition.

The cyclic ureides may be formed into pharmaceutically acceptable salts. Pharmaceutically acceptable salts of the compounds of the invention and physiologically functional derivatives thereof include salts derived from an appropriate base, such as an alkali metal (for example, sodium, potassium), an alkaline earth metal (for example, calcium, magnesium), ammonium and NX₄⁺(wherein X is C₁-C₄ alkyl). Pharmaceutically acceptable salts of an amino group include salts of organic carboxylic acids, such as tartaric, aliphatic, cycloaliphatic, aromatic, heterocyclic, carboxylic and sulfonic classes of organic acids, such as, for example, formic, glucuronic, malic, maleic, fumaric, pyruvic, aspartic, glutamic, benzoic, anthranilic, mesylic, salicylic, hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, hydroxybutyric, cyclochexylaminosulfonic, galactaric and galacturonic acid and the like, lactobionic, fumaric, and succinic acids; organic sulfonic acids, such as methanesulfonic, ethanesulfonic, isothionic, benzenesulfonic and p-toluenesulfonic acids; and inorganic acids such as hydrochloric, hydrobromic, hydroiodic, nitric, carbonic, sulfuric, sulfamic and phosphoric acid and the like. Pharmaceutically acceptable salts of a compound having a hydroxy group consist of the anion of said compound in combination with a suitable cation such as Na⁺, NH₄⁺ or NX₄⁺ (wherein X is, for example, a C₁-C₄ alkyl group), Ca⁺⁺, Li⁺, Mg⁺⁺, or K⁺ and zinc or organic salts made from primary, secondary and tertiary amines, cyclic amines, N,N′-dibenzylethylenediamine, chloroprocaine, choline, diethanolamine, ethylenediamine, meglumine (N-methylglucamine) and procaine and the like. All of these salts may be prepared by conventional means from the corresponding compound by reacting, for example, the appropriate acid or base with the compound in free form.

A subject in whom administration of the therapeutic cyclic ureides is an effective therapeutic regimen for a disease or disorder is preferably a mammal, more preferably a human, but can be any animal, including a laboratory animal in the context of a clinical trial or screening or activity experiment employing an animal model. Diseases or conditions in which compounds of the present invention are useful include, convulsions, seizures, muscle stiffness, nervous stress, anxiety (U.S. Pat. No. 4,628,056), neuroprotection for the treatment or prevention of damage resulting from cerebral ischemia, head trauma and other acute neurological injury (commonly owned U.S. Pat. No. 6,756,379) and movement disorders such as essential tremor and Parkinson's disease (U.S. Pat. No. 7,166,610).

In general, while the effective dosage of cyclic ureides of the present invention for therapeutic use may vary widely depending on the specific application, e.g., treatment of movement disorders, convulsions, seizures or anti-anxiety, the effective dosage is readily determinable within the skill of the art. Suitable effective doses of the cyclic ureides and pharmaceutical compositions containing them will broadly be in the range of 10 micrograms (μg) to 150 milligrams (mg) per kilogram body weight of the subject per day; preferably the dosage is in the range of 50 μg to 130 mg per kilogram body weight per day, and most preferably in the range of 100 μg to 120 mg per kilogram body weight per day. The desired dose may be presented as one or more sub-dose(s) administered at appropriate intervals throughout the day, or alternatively in a single dose, preferably for morning or evening administration. These daily doses or sub-doses may be administered in unit dosage forms, for example, containing from about 150 mg to about 1500 mg, preferably from about 200 mg to about 1200 mg, more preferably from about 250 mg to about 850 mg, and most preferably about 450 mg of active ingredient per unit dosage form to be administered daily or twice daily. In specific embodiments, the daily dosage is equal to or greater than about 200, 250, 300, 350, 400, 450 mg of active ingredient per unit dosage form to be administered daily or twice daily. Typically, less than about 1500 mg of active ingredient per unit dosage form or preferably less than about 1200 mg is to be administered daily. Alternatively, if the condition of the recipient so requires, the doses may be administered as a continuous or pulsatile infusion. The duration of treatment may be decades, years, months, weeks, or days, as long as the benefits persist.

It is appreciated that the effective dose(s) may vary depending on the patient's age, sex, physical condition, duration and severity of symptoms, duration and severity of the underlying disease or disorder if any, and responsiveness to the administered compound. Accordingly, the foregoing ranges are provided only as guidelines and subject to optimization; however, because of the good tolerability and low toxicity of the compounds 32308-250320 of the present invention, higher doses may be administered. The mode of administration and dosage forms is closely related to the therapeutic amounts of the compounds or compositions which are desirable and efficacious for the given treatment application. Suitable dosage forms include but are not limited to oral, rectal, sub-lingual, mucosal, nasal, ophthalmic, subcutaneous, intramuscular, intravenous, transdermal, spinal, intrathecal, intra-articular, intra-arterial, sub-arachinoid, bronchial, lymphatic, and intra-uterile administration, and other dosage forms for systemic delivery of active ingredients. The pharmaceutical composition of the present invention can be administered orally in the form of tablets, pills, capsules, caplets, powders, granules, suspension, gels and the like. Oral compositions can include standard vehicles, excipients, and diluents. The oral dosage forms of the present pharmaceutical composition can be prepared by techniques known in the art and contain a therapeutically effective amount of the cyclic ureide of the present invention.

Formulations suitable for oral administration are preferred. To prepare such pharmaceutical dosage forms, one or more of the aforementioned compounds of formula (1) are intimately admixed with a pharmaceutical carrier according to conventional pharmaceutical compounding techniques. The carrier may take a wide variety of forms depending on the form of preparation desired for administration. In preparing the compositions in oral dosage form, any of the usual pharmaceutical media may be employed. Thus, for liquid oral preparations, such as, for example, suspensions, elixirs and solutions, suitable carriers and additives include water, glycols, oils, alcohols, flavoring agents, preservatives, coloring agents and the like. For solid oral preparations such as, for example, powders, capsules and tablets, suitable carriers and additives include starches, sugars, diluents, granulating agents, lubricants, binders, disintegrating agents and the like. Due to their ease in administration, tablets and capsules represent a preferred oral dosage. If desired, tablets may be sugar coated or enteric coated by standard techniques. Preferred cyclic ureides for preparing the oral pharmaceutical formulations include, DMMDPB, MMMDPB or DPB.

The compositions of the present invention can be provided in unit dosage form, wherein each dosage unit, e.g., a teaspoon, tablet, capsule, solution, or suppository, contains a predetermined amount of the active drug or prodrug, alone or in appropriate combination with other pharmaceutically-active agents. The term “unit dosage form” refers to physically discrete units suitable as unitary dosages for human and animal subjects, each unit containing a predetermined quantity of the composition of the present invention, alone or in combination with other active agents, calculated in an amount sufficient to produce the desired effect, in association with a pharmaceutically-acceptable diluent, carrier (e.g., liquid carrier such as a saline solution, a buffer solution, or other physiological aqueous solution), or vehicle, where appropriate.

In the methods of the present invention, the cyclic ureides of the present invention may be administered within a defined time interval before or after ingestion of food. In the case where the cyclic ureides are administered before ingestion of food, the food may be ingested anywhere from immediately after (i.e., within 1-2 minutes) administration of the cyclic ureides to about four (4) hours after administration of the cyclic ureides. Administration may also occur after ingestion of food. In that case, administration of the cyclic ureide may occur anywhere from immediately after (i.e., within 1-2 minutes) ingestion of the food to about 4 hours after ingestion of food. In one embodiment, the food is ingested about thirty (30) minutes before administration of the pharmaceutical composition containing the cyclic ureide. In another embodiment, the food is ingested about sixty (60) minutes before administration of the pharmaceutical composition containing the cyclic ureide. In yet a third embodiment, the food is ingested one hundred twenty (120) minutes or two hours before administration of the pharmaceutical composition containing the cyclic ureide.

Alternatively, the pharmaceutical composition containing the cyclic ureide is administered about thirty (30) minutes, sixty (60) minutes or one hundred twenty (120) minutes (2 hours) before ingestion of the food.

In a preferred embodiment, the food is solid with sufficient bulk and fat content that it is not rapidly dissolved and absorbed in the stomach. More preferably, the food is a meal, such as breakfast, lunch or dinner.

Treatment methods of the present invention using formulations suitable for oral administration may be presented as discrete units such as capsules, cachets, tablets, or lozenges, each containing a predetermined amount of the cyclic ureide of the present invention as a powder or granules. Optionally, a suspension in an aqueous liquor or a non-aqueous liquid may be employed, such as a syrup, an elixir, an emulsion, or a draught. In such pharmaceutical dosage forms, the active agent preferably is utilized together with one or more pharmaceutically acceptable carrier(s) therefore and optionally any other therapeutic ingredients. The carrier(s) must be pharmaceutically acceptable in the sense of being compatible with the other ingredients of the formulation and not unduly deleterious to the recipient thereof.

A tablet may be made by compression or molding, or wet granulation, optionally with one or more accessory ingredients. Compressed tablets may be prepared by compressing the powder in a suitable machine, with the active compound being in a free-flowing form such as a powder or granules which optionally is mixed with a binder, disintegrant, lubricant, inert diluent, surface active agent, or discharging agent. Molded tablets comprised of a mixture of the powdered cyclic ureide together with a suitable carrier may be made by molding in a suitable machine.

A syrup may be made by adding the active compound to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol. The formulations may be presented in unit-dose or multi-dose form.

Nasal and other mucosal spray formulations (e.g. inhalable forms) can comprise purified aqueous solutions of the active compounds with preservative agents and isotonic agents. Such formulations are preferably adjusted to a pH and isotonic state compatible with the nasal or other mucous membranes. Alternatively, they can be in the form of finely divided solid powders suspended in a gas carrier. Such formulations may be delivered by any suitable means or method, e.g., by nebulizer, atomizer, metered dose inhaler, or the like.

In addition to the aforementioned ingredients, formulations of this invention may further include one or more accessory ingredient(s) selected from diluents, buffers, flavoring agents, binders, disintegrants, surface active agents, thickeners, lubricants, preservatives (including antioxidants), and the like. The formulation of the present invention can have immediate release, sustained release, delayed-onset release or any other release profile known to one skilled in the art.

The invention also comprises an article of manufacture which is a container holding the pharmaceutical composition which comprises the cyclic ureide associated with printed labeling instructions. The printed labeling provides that the pharmaceutical composition should be administered within a defined period of time either before or after ingestion of food. The time periods for administration of the pharmaceutical composition either before or after ingestion of food have been set forth above. The composition will be contained in any suitable container capable of holding and dispensing the dosage form and which will not significantly interact with the composition. The labeling instructions will be consistent with the methods of treatment described herein. The labeling may be associated with the container by any means that maintain a physical proximity of the two, by way of non-limiting example, they may both be contained in a packaging material such as a box or plastic shrink wrap or may be associated with the instructions being bonded to the container such as with glue that does not obscure the labeling instructions or other bonding or holding means.

Commonly owned U.S. Pat. No. 6,939,873 (Gutman et al.), issued Sep. 6, 2005, is incorporated herein by reference in its entirety.

In another embodiment, compounds used according to the present invention encompass the family of barbituric acid anticonvulsant compounds and derivatives and structural analogs having the general Formula 1, and salts thereof

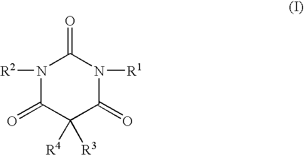

wherein R¹ and R² may be the same or different and are independently hydrogen; lower alkyl, optionally substituted by lower cycloalkyl, acyl, acyloxy, aryl, aryloxy, lower alkoxy, thioalkyl or thioaryl, amino, alkylamino, dialkylamino, or one or more halogen atoms;
phenyl;
CH₂XR⁵, wherein X is S or O and R⁵ is lower alkyl, aryl, or alkylaryl (e.g., benzyl); C(S)XR⁶ or C(O)XR⁶, wherein X is as defined above and R⁶ is lower alkyl or aryl; CXR⁷, wherein X is as defined above and R⁷ is hydrogen, lower alkyl or aryl; and CH(XR⁸)₂, wherein X is as defined above and R⁸ is a lower alkyl group, optionally with the proviso that at least one of R¹ and R² is not hydrogen.

R³ and R⁴ may be the same or different and are independently hydrogen; aryl optionally containing one or more heteroatoms selected from the group consisting of N, S and O; lower acyloxy; phenyl; phenyl substituted with a halogen, lower alkyl group, lower acyl group or derivative thereof or acetamido; benzyl; benzyl substituted on the ring by one or more halogens, lower alkyl groups or both; cycloalkyl, which optionally contains one or more heteroatoms selected from the group consisting of N, O and S; lower alkyl; or lower alkyl substituted with an aromatic moiety. At least one of R³ and R⁴ is an aromatic ring or an aromatic ring containing moiety. As used herein, lower alkyl refers to a branched or straight chain alkyl group having eight or fewer carbons. Alkyl also includes hydrocarbon groups having one or two double or triple bonds in the chain. The embodiment also includes salts of the aforementioned compounds. For compounds and salts of the embodiment,

1. when R¹ and/or R² is methoxymethyl, R³ and R⁴ are not both phenyl, are not both phenyl substituted by lower alkyl, and are not both phenyl substituted by halogen; and

2. when one of R³ and R⁴ is phenyl or benzyl, the other of R³ and R⁴ is not ethyl; and

3. when at least one of R¹ and R² is benzyl, then when one of R³ and R⁴ is phenyl, the other is not allyl; and

4. when one of R¹ and R² is methyl and the other is hydrogen, then when one of R³ and R⁴ is phenyl, the other of R³ and R⁴ is not unsubstituted lower alkyl; and

5. when R¹═R²=R^{a}, Where R^{a} is alkoxymethyl or (acyloxy)methyl, then when one of R³ and R⁴ is 1-phenylethyl, the other of R³ and R⁴ is not propionyloxy.

Furthermore, the following compounds are not included within the scope of the embodiment with respect to compositions, but can be used in practicing the method of the invention.

a) 1-methyl-5-(1-phenylethyl)-5-propionyloxy-barbituric acid,

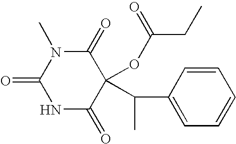

b) 1,3-diphenyl-5,5-(dibenzyl) barbituric acid,

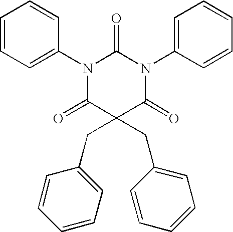

c) 1,3,5-triphenyl barbituric acid, and

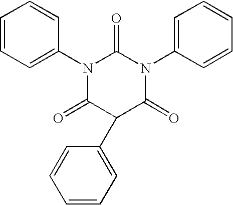

d) 5-benzyl-1,3-dimethyl barbituric acid.

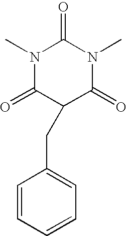

Compounds according to an embodiment of the present invention include lithium 5,5-diphenyl barbiturate, sodium 5,5-diphenyl barbiturate, potassium 5,5-diphenyl barbiturate, and derivatives thereof, presented in the commonly owned U.S. patent application Ser. No. 11/201,024, filed Aug. 10, 2005, published on Jun. 8, 2006 as U.S. Published Patent Application No. US 2006-0122208.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

The embodiments illustrated and discussed in this specification are intended only to teach those skilled in the art the best way known to the inventors to make and use the invention. Nothing in this specification should be considered as limiting the scope of the present invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

The following examples illustrate various aspects of the present invention. They are not to be construed to limit the claims in any manner.

### EXAMPLE 1

Early pharmacokinetic studies in dogs indicated that DMMDPB was more completely absorbed when taken with a high fat meal than with customary dog food. Accordingly, a Phase I clinical study was performed in 23 healthy non-smoking males between 19 and 54 years of age to determine the effect of food on bioavailability of DMMDPB, MMMDPB and DPB.

This was a single center, randomized, single dose, open-label, 2-way, food-effect crossover study to compare the rate and extent of absorption of T2000, administered as 4×100 mg capsules (for a total dose of 400 mg), under fasting and fed conditions. Subjects were confined to a clinical research facility from at least 11 hours prior to drug administration until after the 72.0-hour post-dose blood draw, in each period. Single oral doses were separated by a washout period of at least 21 days.

A total of 25 healthy male non-smokers signed the study-specific informed consent form and were confined for Period 1; of these subjects, 23 were dosed and were considered to have enrolled in the study; all of these enrolled subjects completed the study. All subjects met the inclusion and exclusion criteria described in the protocol and were judged eligible for the study, based on medical history, demographic data (including sex, age, body weight [kg], height [cm], and BMI [kg/m2], medication history, physical examination, vital signs (blood pressure, respiratory rate, heart rate, and oral temperature), 12-lead ECG, brief neurological examination (including assessment of mental status, orientation, memory, arithmetic and spelling, eye movement, gait and coordination), opthalmology examination (lamp examination; performed within 3 months prior to the first dose), and clinical laboratory tests (hematology, biochemistry, urinalysis, HIV and hepatitis C [HCV] antibodies, and hepatitis B antigen [HBsAg]). In addition, each subject had a negative urine drug screen at screening. Ages ranged from 19 to 54 years, and subject body mass indices ranged between 18.3 and 29.5 kg/m², at the time of screening. Screening procedures took place within 28 days prior to Period 1 drug administration.

Subjects abstained from food or drink containing xanthine derivatives or xanthine-related compounds (e.g. coffee, tea, caffeine-containing sodas, colas, chocolate, or decaffeinated products) and energy drinks from 48 hours prior to drug administration, until the end of sample collection in each period; alcohol from 24 hours prior to drug administration, until the end of sample collection in each period, and grapefruit products (e.g. fresh, canned, or frozen); natural food supplements (including garlic as a supplement), and vitamins from 7 days prior to drug administration, until the end of sample collection in each period. Any subjects who felt dizzy or drowsy when they left the clinical facility were advised against performing activities requiring mental alertness, judgment, and physical coordination until they felt safe to do so.

Treatment A: The subjects fasted for at least 10 hours prior to drug administration.

Treatment B: After a supervised overnight fast of at least 10 hours, and 30 minutes before drug administration, subjects were served a standard high-fat, high-caloric breakfast of approximately 1000 calories (approximately 150 calories from protein, 250 calories from carbohydrates, and 500 calories from fat). The breakfast consisted of two pats of butter, two large eggs fried in butter, 64 gm of toast, 32 gm of bacon, 128 gm of hash brown potatoes, 200 ml of whole milk. Subjects were required to completely consume this breakfast within 30 minutes, and prior to drug administration. Subjects were dosed on the momrings of Apr. 20, 2003, and May 11, 2003, between 07:00 AM and 07:48 AM. Subjects were administered the test or reference medication as a single oral dose of 4 capsules, each containing 100 mg of prodrug T2000 (total dose of 400 mg), with approximately 240 ml of water. Subjects were dosed as specified in the protocol, and subsequently fasted for a period of at least 4 hours. A mouth and hand check was performed to ensure the subjects had swallowed the study medication.

All blood samples were drawn into blood collection tubes (1×7 ml) containing EDTA K3 prior to drug administration and 0.500, 1.00, 2.00, 3.00, 4.00, 5.00, 6.00, 8.00, 10.0, 12.0, 15.0, 18.0, 24.0, 36.0, 48.0, 72.0, 96.0, and 120 hours post-dose in each period. All blood samples were collected via direct venipuncture. The total volume of blood drawn from each subject completing this study did not exceed 301 ml.

Analysis of T-2000, MMMDPB and DPB were performed using High Performance Liquid Chromatographic Method with Tandem Mass. Spectrometry Detection. The analytical method was developed and validated.

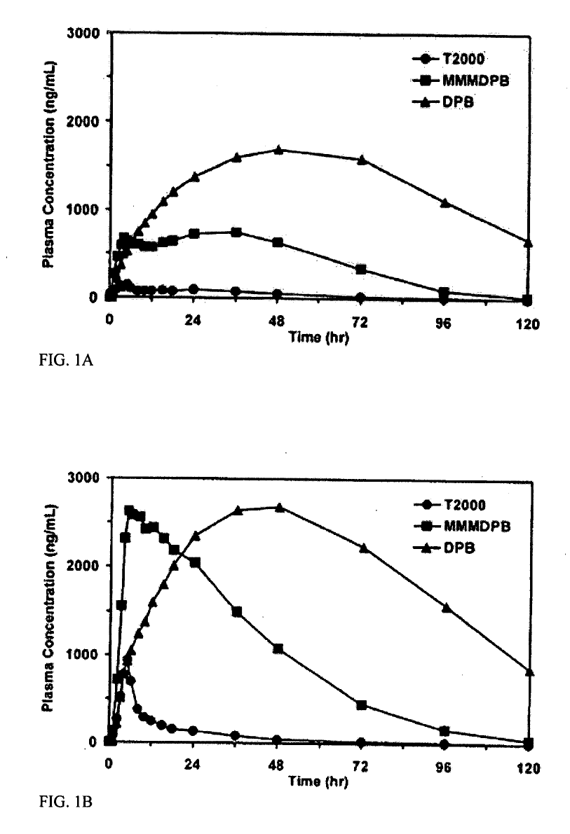

Each subject received a single 400 mg oral dose of DMMDPB under fed and under fasting conditions. There was a 3-week washout period between doses. DMMDPB was well tolerated. Adverse events reported were minor (total of 35) and occurred in both fed and fasting conditions. The rate and extent of absorption of DMMDPB as well as MMMDPB and DPB increased when administered after a high fat, high calorie breakfast consisting of two (2) pats of butter, two large eggs fried in butter, 64 g of toast, 32 g of bacon, 128 g of hash browns, and 200 ml of whole milk. (FIG. 1, Table 1). The ratios (fasting/fed) for geometric mean AUC₀₋ₜ were 35.6% (DMMDPB), 36.6% (MMMDPB) and 65.3% (DPB). The ratios (fasting/fed) for geometric mean Cₘₐₓ were 14.6% (DMMDPB), 31.9% (MMMDPB) and 62.9% (DPB).

TABLE 1
Mean Pharmacokinetic Parameters, Ratios and 90% Confidence
Intervals for DMMDPB, MMMDPB and DPB After Single Oral Doses
of DMMDPB in Healthy Subjects in the Fasting and Fed State (n = 23, 400 mg)

| | | Treatment | | Ratio (%) | |
|---|---|---|---|---|---|
| Analyte | Metric | Fasting | Fed | (Fasting/Fed) | 90% CI (%) |
| DMMDPB | AUC₀₋ₜ (ng-hr/mL) | 3098.58 | 8712.22 | 35.57 | 28.43-44.49 |
| | AUC_{0-∞}* (ng-hr/mL) | 5320.07 | 10755.24 | 49.46 | |
| | Cₘₐₓ (ng/mL) | 129.34 | 887.14 | 14.58 | 41.51-58.95 |
| | Tₘₐₓ (hr) | 7.52 | 4.88 | 154.34 | |
| | T_{1/2}* (hr) | 26.13 | 18.46 | 141.55 | 11.92-17.83 |
| MMMDPB | AUC₀₋ₜ (ng-hr/mL) | 36364.43 | 99477.88 | 36.56 | 31.46-42.48 |
| | AUC_{0-∞}** (ng-hr/mL) | 53956.88 | 120283.39 | 44.86 | |
| | Cₘₐₓ (ng/mL) | 898.20 | 2818.96 | 31.86 | 38.74-51.94 |
| | Tₘₐₓ (hr) | 20.2 | 8.83 | 229.02 | |
| | T_{1/2}** (hr) | 26.80 | 16.21 | 165.33 | 27.51-36.90 |
| DPB | AUC₀₋ₜ (ng-hr/mL) | 143037.24 | 219124.98 | 65.28 | 59.81-71.25 |
| | AUC_{0-∞} (ng-hr/mL) | 184514.06 | 271169.43 | 68.04 | |
| | Cₘₐₓ (ng/mL) | 1753.19 | 2786.10 | 62.93 | 59.76-77.47 |
| | Tₘₐₓ (hr) | 54.8 | 48.3 | 113.50 | |
| | T_{1/2} (hr) | 43.03 | 38.55 | 111.62 | 58.21-68.02 |

Least-squares geometric mean (AUC₀₋ₜ, AUC_{0-∞});
Arithmetic mean (Cₘₐₓ, Tₘₐₓ, T_{1/2});
*n = 18;
**n = 20

Pharmacokinetic parameters were calculated using Bioequiv (release 3.40), a proprietary software developed and validated for bioequivalence studies at Anapharm Inc. This software performs non-compartmental analyses of pharmacokinetic parameters and statistical analyses (via SAS release 6.12) according to FDA, HPFB and EMEA guidance. The mean, standard deviation (SD), coefficient of variation (CV (%)) and range (min and max.) were calculated for plasma concentrations of T2000, MMMDPB and DPB for each sampling time and treatment. As well, the mean, SD, CV (%) and range were calculated for the AUC₍₀₋ₜ₎ (ng h/ml), AUC_{0-inf} (ng-h/ml), Cₘₐₓ(ng/ml), Tₘₐₓ (h), T_{1/2el} (h), Kₑₗ (h), TLIN (h) and LQCT (h). The calculation of these pharmacokinetic parameters is explained below. Areas Under the Concentration-Time Curves: AUC₀₋ₜ was calculated using the linear trapezoidal rule.

The AUC_{0-inf} was calculated as:

$${AUC}_{0 - t} + \frac{{C}_{t}}{{K}_{el}}$$

Where: Cₜ=the last observed non-zero concentration, AUC₀₋ₜ=the AUC from time zero to the time of the last non-zero concentration, and Kₑₗ=the elimination constant. The maximum observed concentration and time of observed peak concentration were determined as follows. Cₘₐₓ, the maximum observed concentration, and Tₘₐₓ, the time to reach that peak concentration, were determined for each subject and for each treatment.

The half-life and elimination rate constant were determined as follows. The elimination rate constant (K_{cl}), regression analyses were performed on the natural log(Ln) of plasma concentration values (y) versus time (x). Calculations were made between TLIN and LQCT (see definitions below). The Kₑₗ was taken as the slope multiplied by (−1) and the apparent half-life as (In 2)/Kₑₗ.

TLIN and LQCT: TLIN, the time point where Ln-linear Kₑₜ calculation begins, and LQCT, the sampling time of the last quantifiable concentration used to estimate the Kₑₗ, were determined by the pharmacokinetic scientist (according to Anapharm's standard operating procedures) for each subject and for each treatment. At least 4 non-zero observations during the terminal elimination phase were used to calculate the Kc. A minimum of 3 observations was used if less than 4 observations were available. When the constant (Kₑₗ) could not be determined (e.g.: less than 3 non-zero concentrations available in the terminal elimination phase or the correlation coefficient (r value) from the regression of the in-linear elimination phase was less than 71%), then data from these subjects were not used to calculate the AUC_{0-inf}, T_{1/2el}, Kₑₗ and Residual area, but were used in all other calculations.

For T2000, MMMDPB and DPB, analysis of variance was performed on the intransformed data of AUC₀₋ₜ, AUC_{0-inf} and Cₘₐₓ. ANOVA was also carried out on the untransformed data of Tₘₐₓ, T_{1/2} and Kₑₗ. All ANOVAs were performed with the SAS General Linear Models Procedure (GLM). The model included sequence, subject within sequence, period and treatment as factors. All sums of squares (Types I, II, III and IV) were reported. The sequence effect was tested using the subjects within sequence effect as the error term. The treatment and period effects were tested against the residual mean square error. Probability (p) values were derived from Type III sums of squares. For all analyses, effects were considered statistically significant if the probability associated with ‘F’ was less than 0.050. Based on the pairwise comparisons of the In-transformed AUC₀₋ₜ, AUC_{0-inf} and Cₘₐₓ data, the ratios of the least-squares means, calculated according to the formulae “e^{(x-y)} X 100”, as well as the 90% geometric confidence intervals were determined. Detectable differences were calculated (using the t-test method) to determine the percentage difference between formulations in AUC₀₋ₜ, AUC_{0-inf} and Cₘₐₓ that could be detected in the study with an alpha level of 0.05 and a beta level of 0.20. The power to detect a difference of at least 20% between the formulations with an alpha level of 0.05 was also presented for these parameters. Finally, the intra-subject CVs were also calculated.

We claim:

1. A method of improving bioavailability of a pharmaceutical composition comprising, administering a therapeutically effective amount of at least one compound according to the following formula: 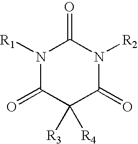
to a mammal, wherein R₃ and R₄ are each independently selected from the group consisting of lower alkyl, phenyl and lower alkyl substituted phenyl, and R₁ and R₂ are each independently either a hydrogen atom or a radical of the formula 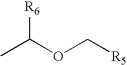
wherein R₅ and R₆ are each independently selected from the group consisting of H, lower alkyl, phenyl and lower alkyl substituted phenyl, its pharmaceutically acceptable salts and prodrugs thereof and at least one pharmaceutically acceptable excipient, wherein the pharmaceutical composition is administered from about 4 hours prior to ingestion of food to about 4 hours after ingestion of food.

2. The method of claim 1 wherein, the food is ingested from about 2 hours to about 5 minutes before administration of the pharmaceutical composition.

3. The method of claim 2 wherein, the food is ingested from about 30 minutes to about 5 minutes before administration of the pharmaceutical composition.

4. The method of claim 1 wherein, the food is ingested from about 5 minutes to about 2 hours after administration of the pharmaceutical composition.

5. The method of claim 4 wherein, the food is ingested from about 5 minutes to about 30 minutes after administration of the pharmaceutical composition.

6. The method of claim 1 wherein, the fasting/fed ratios for the geometric mean AUC₀₋ₜ, of DMMDPB is at least about 30%.

7. The method of claim 1 wherein, the fasting/fed ratios for the geometric mean AUC₀₋ₜ of MMMDPB is at least about 30%.

8. The method of claim 1 wherein, the fasting/fed ratios for the geometric mean AUC₀₋ₜ of DPB is at least about 60%

9. The method of claim 1 wherein the fasting/fed ratios for the geometric mean AUC₀₋ₜ for DMMDPB is at least about 35.0%, MMMDPB is at least about 36% and DPB is at least about 65%.

10. The method of claim 9 wherein the geometric mean Cₘₐₓ is at least about 14.0% for DMMDPB, at least about 31.0% for MMMDPB and at least about 62.0% for DPB.

11. The method of claim 1 wherein, R₅ and R₆ are each independently H or lower alkyl.

12. The method of claim 1 wherein, R₃ and R₄ are each independently phenyl.

13. The method of claim 1 wherein, at least one of R₁ and R₂ is defined by the formula 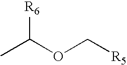
wherein R₅ and R₆ are each independently selected from H or lower alkyl.

14. The method of claim 1 wherein, at least one of R₁ and R₂ is —CH₂OCH₃.

15. The method of claim 1 wherein, both R₅ and R₆ are phenyl, R₁ is —CH₂OCH₃ and R₂ is H.

16. The method of claim 1 wherein, both R₅ and R₆ are phenyl and both R₁ and R₂ are —CH₂OCH₃.

17. The method of claim 1 wherein, the pharmaceutical composition comprises a tablet.

18. The method of claim 1 wherein, the pharmaceutical composition comprises a capsule.

19. The method of claim 1 wherein, the mammal is a human.

20. An item of manufacture comprising, a container containing a pharmaceutical composition of a therapeutically effective amount of at least one 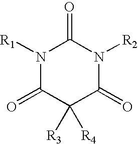
compound according to the following formula:
wherein R₃ and R⁴ are each independently selected from the group consisting of lower alkyl, phenyl and lower alkyl substituted phenyl, and R₁ and R₂ are each independently either a hydrogen atom or a radical of the formula 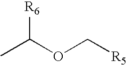
wherein R₅ and R₆ are each independently selected from the group consisting of H, lower alkyl, phenyl and lower alkyl substituted phenyl, its pharmaceutically acceptable salts and prodrugs thereof and at least one pharmaceutically acceptable excipient, wherein the container is associated with printed labeling advising that food should be ingested from about 2 hours prior to administration of the pharmaceutical composition to about 2 hours after administration of the pharmaceutical composition.

21. The item of manufacture of claim 20 wherein, the pharmaceutical composition is a tablet unit dosage form.

22. The item of manufacture of claim 20 wherein, the pharmaceutical composition is a capsule unit dosage form.

23. A method of improving bioavailability of a pharmaceutical composition comprising, administering a therapeutically effective amount of at least one compound according to the following formula to a mammal: 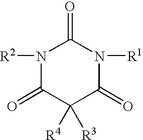
wherein R¹ and R² may be the same or different and are independently
lower alkyl, substituted by lower cycloalkyl, acyl, acyloxy, aryl, aryloxy, thioalkyl or thioaryl, amino, alkylamino, dialkylamino, or one or more halogen atoms;
phenyl;
C(O)XR⁶, wherein X is S or O and R⁶ is lower alkyl or aryl;
CXR⁷, wherein X is as defined above and R⁷ is hydrogen, lower alkyl or aryl; and
CH(XR⁸)₂, wherein X is as defined above and R⁸ is a lower alkyl group; and
wherein
R³ and R⁴ may be the same or different and are independently hydrogen;
aryl optionally containing one or more heteroatoms selected from the group consisting of N, S, and O;
lower acyloxy;
phenyl substituted with lower acyl group or derivative thereof or acetamide;
benzyl; benzyl substituted on the ring by one or more halogens, lower alkyl groups or both; cycloalkyl, which optionally contains one or more heteroatoms selected from the group consisting of N, O, and S;
lower alkyl; or lower alkyl substituted with an aromatic moiety;
provided that at least one of R³ and R⁴ is an aromatic ring or an aromatic ring containing moiety,
and salts thereof,
with the proviso that:
when one of R³ and R⁴ is benzyl, the other of R³ and R⁴ is not ethyl, the compound is other than
a) 1-methyl-5-(1-phenylethyl)-5-propionyloxy-barbituric acid,
b) 1,3-diphenyl-5,5-(dibenzyl) barbituric acid,
c) 1,3,5-triphenyl barbituric acid, and
d) 5-benzyl-1,3-dimethyl barbituric acid
and at least one pharmaceutically acceptable excipient, wherein the pharmaceutical composition is administered from about 4 hours prior to ingestion of food to about 4 hours after ingestion of food.

24. A method of improving bioavailability of a pharmaceutical composition comprising, administering a therapeutically effective amount of sodium 5,5-diphenyl barbiturate or a derivative thereof and at least one pharmaceutically acceptable excipient, wherein the pharmaceutical composition is administered from about 4 hours prior to ingestion of food to about 4 hours after ingestion of food.
